(19) United States
(12) Patent Application Publication
(10) Pub. No.: US 2024/0365780 A1
(43) Pub. Date: Nov. 7, 2024

(54) MICROBIAL INOCULANT COMPOSITIONS AND METHODS

(71) Applicant: RAISON, LLC, SIOUX FALLS, SD (US)

(72) Inventor: Tony Hagen, Sioux Falls, SD (US)

(21) Appl. No.: 18/777,004

(22) Filed: Jul. 18, 2024

Related U.S. Application Data
(63) Continuation of application No. 18/647,232, filed on Apr. 26, 2024.
Application No. 18/647,232 is a continuation of application No. 16/635,149, filed on Jan. 29, 2020, now Pat. No. 11,980,183.
(60) Provisional application No. 62/541,422, filed on Aug. 4, 2017.
(60) Provisional application No. 62/660,836, filed on Apr. 20, 2018.
(60) Provisional application No. 62/663,067, filed on Apr. 26, 2018.
(60) Provisional application No. 62/672,190, filed on May 16, 2018.

(51) Int. Cl. A01N 25/02; A01H 5/10; A01N 57/12; A01N 59/26

(52) U.S. Cl. CPC A01N 25/02; A01H 5/10; A01N 57/12; A01N 59/26

(57) ABSTRACT

A microbial inoculant composition includes aquatic bacterial species. In some embodiments, the microbial inoculant composition includes at least one of an aquatic Pseudomonas spp. and a Clostridium spp.

### CROSS-REFERENCE TO RELATED APPLICATION

This application is a continuation of U.S. patent application Ser. No. 18/647,232, filed Apr. 26, 2024, which is a continuation of U.S. patent application Ser. No. 16/635,149, filed Jan. 29, 2020 (now U.S. Pat. No. 11,980,183), which is a National Stage Entry of International Application No. PCT/US2018/045215, filed Aug. 3, 2018, which claims the benefit of priority to U.S. Provisional Patent Application No. 62/541,422, filed Aug. 4, 2017; U.S. Provisional Patent Application No. 62/660,836, filed Apr. 20, 2018; U.S. Provisional Patent Application No. 62/663,067, filed Apr. 26, 2018; and U.S. Provisional Patent Application No. 62/672,190, filed May 16, 2018. The content of each application is incorporated herein by reference in its entirety.

### SUMMARY

This disclosure describes, in one aspect, a microbial inoculant composition. Generally, the microbial inoculant composition includes an aquatic Pseudomonas spp. and a Clostridium spp., wherein the aquatic Pseudomonas spp. produces a plant hormone beneficial to a plant.

In some embodiments, the aquatic Pseudomonas spp. is P. moraviensis or P. fluorescens.

In some embodiments, the Clostridium spp. is Clostridium saccharobutylicum.

In some embodiments, the microbial inoculant composition further includes a Bacillus spp. In some of these embodiments, the Bacillus spp. is B. megaterium, B. subtilis, or B. licheniformis.

In some embodiments, the microbial inoculant composition further includes an aquatic Delftia spp.

In some embodiments, the microbial inoculant composition further includes an aquatic Chryseobacterium spp.

In some embodiments, the microbial inoculant composition further includes Brevundimonas kwangchunensis, Fictibacillus barbaricus/Bacillus barbaricus, a Prosthecobacter spp., Sphingobacterium multivorum, or a Sphingomonas spp.

In some embodiments, the microbial inoculant composition further includes Bacillus megatcrium, Bacillus ainyloliquilaciens, Bacillus subtilits, Bacillus punthus, Sphingosinicella microcystinivorans, Pseudomonas chlororaphis, Pseudomonas mandelii, Pseudomonas umsongensis, a Clostridium spp., Arthrobacter ramosus, Streptomyces yogyakartansis, an Arthrobacter spp., a Xanthomonas spp., or Chryseobacterium indologenes.

In another aspect, this disclosure describes a plant that includes adhered to at least a portion of the plant.

In another aspect, this disclosure describes a seed having any embodiment of the microbial inoculant composition summarized above adhered to at least a portion of the seed.

In another aspect, this disclosure describes a method that includes applying any embodiment of the microbial inoculant composition summarized above to a tissue of a plant.

In another aspect, this disclosure describes a method that includes applying any embodiment of the microbial inoculant composition summarized above to a surface of a seed.

In another aspect, this disclosure describes a method that includes applying any embodiment of the microbial inoculant composition summarized above to a seed bed.

In another aspect, this disclosure describes a method that includes applying any embodiment of the microbial inoculant composition summarized above to a field comprising a plurality of plants.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Effect of microbial inoculant composition on soybeans. Soybeans in the middle and right are treated with an exemplary microbial composition. Soybeans on the left are untreated. Treated soybeans have bigger leaves and more branching.

FIG. 2. Effect of microbial inoculant composition on soybeans. Soybeans on the left are treated with an exemplary microbial composition. Soybeans on the right are untreated. Treated soybeans show reduced leaf wilt and greater leaf size.

FIG. 3. Effect of microbial inoculant composition on wheat. Wheat on the right is untreated. Wheat in the middle is treated at the foliar stage with an exemplary microbial inoculant composition. Wheat on the left is treated with the same microbial inoculant composition at the seed coat stage. Untreated wheat averaged 29 heads, foliar-treated wheat averaged 40 heads, seed-coat-treated wheat averaged 61 heads.

FIG. 4. Effect of microbial inoculant composition on canola. Canola on the right was untreated. Canola on the left was treated with an exemplary microbial inoculant composition. Treated canola shows increased branching and increased pod number.

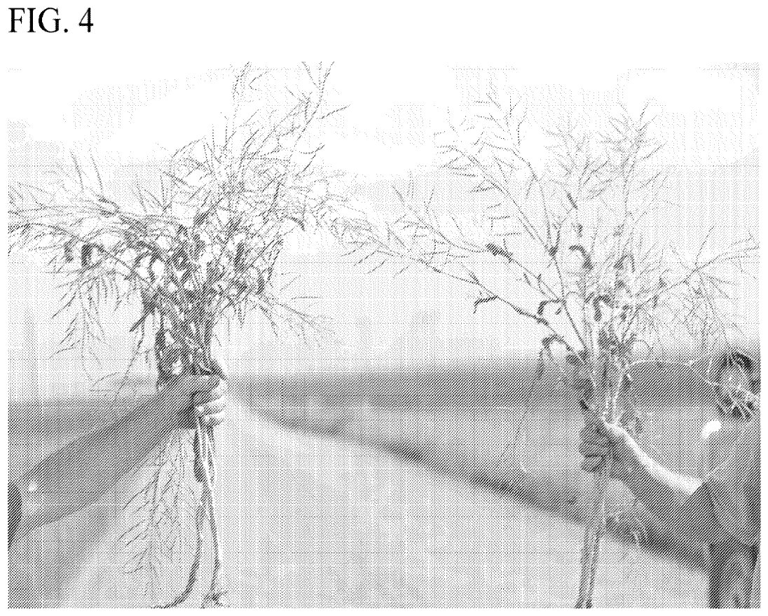

FIG. 5. Effect of microbial inoculant composition on corn. Upper cob is from an untreated plant. Lower cob is from a plant treated with an exemplary microbial inoculant composition. The lower cob shows an increase in kernels per ring compared to the untreated cob. Difference in size of the kernels is due to the treated corn being at an earlier stage of development than the untreated corn.

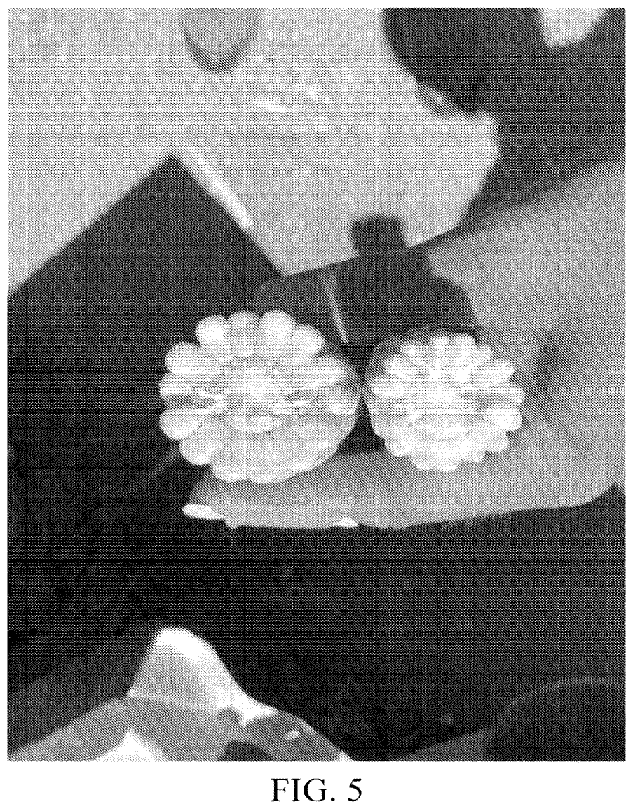

FIG. 6. Sunflower subjected to mower damage, then treated with an exemplary microbial inoculant composition.

FIG. 7. Cabbage subjected to mower damage, then treated with an exemplary microbial inoculant composition.

### DETAILED DESCRIPTION OF ILLUSTRATIVE EMBODIMENTS

This disclosure describes microbial inoculant compositions that include aquatic microbial species for application to terrestrial plants. In some embodiments, the inoculant mixture also includes a species that produces and/or maintains a microenvironment in the plant that is suitable for other microbes in the inoculant mixture to thrive.

A loss of biodiversity within a soil matrix can lead to yield depression of agricultural crops. Microbial inoculants can increase solubilization, uptake, and/or assimilation of nutrients such as, for example, carbon, nitrogen, potassium. phosphorus, selenium, cobalt, zinc, and copper. Microbial inoculants also can reduce plant pathogen damage to crops and provide a stable and continuous source of plant hormones that enhance growth. While microorganisms capable of promoting plant growth and plant production can occur naturally in soil, the mere presence of the microbes does not guarantee the successful integration of the microbes.

This disclosure describes novel microbial inoculant compositions isolated from an aquatic environment for application to terrestrial plants. In some embodiments, the inoculant mixture also includes a species that produces and/or maintains a microenvironment in the plant that is suitable for other microbes in the inoculant mixture to thrive.

Generally, the microbial inoculant composition includes a Pseudomonas spp. and a Clostridium spp., such as, for example, P. fluorescens and C. saccharobutylicum.

In some embodiments, the microbial inoculant composition further includes one or more of Agrobacterium tumefaciens (TPD7005), Bacillus megaterium (TPD7007), Bacillus megaterium (TPD 7008), Agrobacterium rhizogenes (TPD7009), Microbacterium testaceum (TPD7010), Bacillus megaterium (TPD7011), Microbacterium spp. (TPD7012), Pedobacter kribbensis (TPD70013), Janthinobacterium lividum (TPD7014), Bacillus racemilacticus (TPD7015), Bacillus megaterium (TPD 7018), Delftia spp. (TPD3002), Chryseobacterium spp. (TPD3003), Bacillus licheniformis, Brevundimonas kwangchunensis (TPD3004), Fictibacillus barbaricus/Bacillus barbaricus (TPD3005), Prosthecobacter spp. (TPD3006), Lactobacillus plantarum (TPD3007), Sphingobacterium multivorum, Sphingomonas spp. (TPD3009), Sphingosinicella microcystinivorans (TPD3010), Pseudomonas chlororaphis, Pseudomonas mandelii, Pseudomonas umsongensis, Clostridium saccharobutylicum (TPD3014), Arthrobacter ramosus (TPD3015), Streptomyces yogyakartaensis (TPD3016), Arthrobacter spp. (TPD3017), Xanthomonas spp., Chryseobacterium indologenes (TPD3019), or Lactobacillus plantarum.

Table 1 shows 16S RNA analysis and/or whole genome shotgun sequencing project data for exemplary members of the microbial inoculant composition.

TABLE 1

| Species | Designation | GenBank Accession No. |
|---|---|---|
| Pseudomonas veronii | TPD3012 | MH190219.1 |
| Pseudomonas mandelii | TPD3013 | MH221124.1 |
| Pseudomonas moraviensis | TPD3001 | MH190053.1 |
| Pseudomonas protegens | TPD3011 | MH221127.1 |
| Pantoea agglomerans | TPD7001 | MH190052.1 |
| Clostridium saccharobutylicum | TPD3014 | MH189851.1 |
| Clostridium saccharobutylicum | TPD7003 | MH192394.1 |
| Erwinia aphidicola | TPD7004 | MH190220.1 |
| Serratia liquefaciens | TPD7002 | MH190215.1 |
| Pedobacter kribbensis | TPD70013 | MH221086.1 |
| Janthinobacterium lividum | TPD7014 | MH221099.1 |
| Bacillus racemilacticus | TPD7015 | MH221098.1 |
| Sphingomonas spp. | TPD3009 | QDFK00000000.1 |
| Agrobacterium tumefaciens | TPD7005 | QDFL00000000.1 |
| Bacillus megaterium | TPD7018 | QDFM00000000.1 |
| Sphingomonas spp. | TPD3009 | QDFN00000000.1 |
| Bacillus megaterium | TPD7007 | QDFO00000000.1 |
| Bacillus megaterium | TPD7008 | QDFP00000000.1 |
| Arthrobacter spp. | TPD3018 | QDFQ00000000.1 |
| Agrobacterium rhizogenes | TPD7009 | QDFR00000000.1 |
| Sphingomonas melonis | TPD3008 | QDFS00000000.1 |
| Microbacterium testaceum | TPD7010 | QDFT00000000.1 |
| Bacillus megaterium | TPD7011 | QDFU00000000.1 |
| Microbacterium spp. | TPD7012 | QDFV00000000.1 |

Finally, in some embodiments, the microbial inoculant composition further includes one or more of yeast strain TAH3020 or yeast strain TAH3021.

The microbial inoculant composition can promote plant growth (e.g., increase leaf size, increase root mass), decrease the impact of stress, decrease water consumption, increase solubility and/or assimilation of nutrients, increase feed value, increase decay of carbon-containing molecules so that the organic molecules are more readily available to the plant, provide growth hormones to plants, and/or increase plant metabolism (thereby decreasing the time to fruit). Moreover, in legumes, the microbial inoculant composition can increase pod numbers, increase root growth, increase nodulation, and/or increase the number of branches per plant. In some embodiments, the microbial inoculant composition can be applied to contact and/or interact endophytically with the plant.

In particular, bacteria in the microbial inoculant composition can produce 1-aminocyclopronane-1-carboxylate (ACC) deaminase. ACC can lower plant ethylene levels, often a result of various stresses such as, for example, stress to heat and/or drought. ACC can interact synergistically with the plant and bacterial auxin, indole-3-acetic acid (IAA). ACC-producing basteria not only can directly promote plant growth, but also can protect plants against flooding, drought, salt, flower wilting, metals, organic containments, bacterial pathogens, and fungal pathogens.

In particular, decreasing water consumption can increase solubilization of minerals and/or fertilizers so that water requirements are reduced to transport the minerals and/or fertilizers from the roots, increase root development so that soil nutrients can be obtained from a greater area and/or water can be obtained from deeper in the soil, and/or reduce daily heat stress. Reducing daily heat stress allows the plant to better acquire CO.sub.2, thereby metabolize more sugars and increase yield, regulate pH, and/or produce more energy during daylight hours.

The microbial inoculant compositions can include additional microbial species or other additives to provide desired physiological, pesticidal, metabolic, or other activity to the composition.

For example, in certain embodiments the microbial inoculant compositions can include one or more of the following microbial species: an Acetobacteraceae spp. (e.g., Acidisphaera spp.), an Acetivibrio spp. (e.g., Acetivibrio cellulolyticus), an Acidiphilium spp., an Acidimicrobiaceae spp. (e.g., an Acidimicrobium spp., an Aciditerrimonas spp.), an Acidobacteriales spp. (e.g., an Acidobacteriaceae spp. [e.g., an Acidobacterium spp.]), an Acidothermus spp., an Acidovorax spp. (e.g., Acidovorax citrulli), an Acinetobacter spp. (e.g., Acinetobacter lwoffii), an Actinoallomurus spp. (e.g., Actinoallomurus iriomotensis), an Actinocatenispora spp. (e.g., Actinocatenispora rupis), an Actinomadura spp., an Actinomycetales spp. (e.g., an Actinomyces spp.), an Actinoplanes spp. (e.g., Actinoplanes auranticolor), an Actinopolymorpha spp. (e.g., Actinopolymorpha pittospori), an Actinotalea spp. (e.g., Actinotalea fermentans), an Adhaeribacter spp. (e.g., Adhaeribacter terreus), an Aeromicrobium spp. (e.g., Aeromicrobium fastidiosum), an Afipia spp., an Agromyces spp. (e.g., Agromyces ulmi, Agromyces subbeticus), an Alcaligenaceae spp., an Algoriphagus spp., an Alkaliflexus spp., an Alphaproteobacteria spp., an Alsobacter spp. (e.g., Alsobacter metallidurans), an Altererythrobacter spp., an Alteromonadaceae spp., an Amaricoccus spp., an Aminobacter spp., an Amycolatopsis spp. (e.g., Amycolatopsis iriomotensis, Amycolatopsis vancoresmycina), an Anaeromyxobacteraceae spp. (e.g., an Anaeromyxobacter spp. [e.g., Anaeromyxobacter dehalogenans]), an Ancylobacter spp., an Angustibacter spp. (e.g., Angustibacter peucedani), an Aquabacterium spp., an Aquicella spp., an Armatimonadetes spp., an Arenimonas spp. (e.g., Arenimonas oryziterrae), an Arsenicicoccus spp. (e.g., Arsenicicoccus dermatophilus), an Arthrobacter spp. (e.g., Arthrobacter pascens, Arthrobacter tumbae), an Asanoa spp. (e.g., Asanoa ishikariensis), an Azohydromonas spp. (e.g., Azohydromonas australica), an Azonexus spp., an Azospira spp. (e.g., Azospira oryzae), an Azospirillum spp. (e.g., Azospirillum lipoferum), an Azotobacter spp. (e.g., Azotobacter chroococcum), a Bacillaceae spp. (e.g., a Bacillus spp. [e.g., Bacillus acidiceler, Bacillus aphidicola, Bacillus senegalensis, Bacillus megaterium, Bacillus subtilis]), a Bacteroidetes spp. (e.g., a Bacteroidales spp. [e.g., a Bacteroides spp.]), a Bauldia spp. (e.g., Bauldia consociate), a Bdellovibrionaceae spp., a Beijerinckia spp., a Blastococcus spp. (e.g., Blastococcus saxobsidens), a Blastomonas spp., a Bordetella spp. (e.g., Bordetella hinzii), a Bosea spp., a Bradyrhizobiaceae, spp. (e.g., Bradyrhizobium spp. [e.g., Bradyrhizobium elkanii, Bradyrhizobium yuanmingense]), a Brevibacteriaceae spp., a Brevundimonas spp. (e.g., Brevundimonas lenta), a Bryobacter spp., a Burkholderiales spp. (e.g., a Burkholderiaceae spp. [e.g., a Burkholderia spp.]), a Brucellaceae spp., a Buttiauxella spp. (e.g., Buttiauxella izardii), a Byssovorax, spp., a Caldilineales spp. (e.g., a Caldilineaceae spp. [e.g., a Caldilinea spp.]), a Caloramator spp., a Candidatus spp. (e.g., Candidatus brocadiaceae, Candidatus entotheonella, Candidatus koribacter, Candidatus nitrosoarchaeum, Candidatus phytoplasma, Candidatus saccharibacteria, Candidatus solibacter), a Carnobacterium spp., a Catenuloplanes spp., a Catellatospora spp., (e.g., Catellatospora citrea), a Caulobacteraceae spp. (e.g., a Caulobacter spp. [e.g., Caulobacter tundrae]), a Cellulosimicrobium spp. (e.g., Cellulosimicrobium cellulans), a Cellvibrio spp. (e.g., Cellvibrio vulgaris), a Cellulomonas spp. (e.g., Cellulomonas terrae), a Chelatococcus spp. (e.g., Chelatococcus asaccharovorans, a Chitinophagaceae spp., a Chromobacteriaceae spp., a Chloroflexales spp. (e.g., a Chloroflexaceae spp. [e.g., a Chloroflexus spp.]), a Chthoniobacter spp. (e.g., Chthoniobacter flavus), a Chryseobacterium spp., a Citrobacter spp., a Clavibacter spp. (e.g., Clavibacter michiganensis), a Clostridiaceae spp. (e.g., a Clostridium spp. [e.g., Clostridium bowmanii, Clostridium gasigenes, Clostridium uliginosum, Clostridium vincentii]), a Comamonadaceae spp. (e.g., a Comamonas, spp. [e.g., Comamonas koreensis]), a Conexibacteraceae spp. (e.g., a Conexibacter spp. [e.g., Conexibacter woesei]), a Coxiellaceae spp., a Crenotrichaceae spp. a Cryomorphaceae spp., a Cryobacterium spp. (e.g., Cryobacterium mesophilum), a Cupriavidus spp. (e.g., Cupriavidus campinensis), a Curtobacterium spp., a Cyanobacteria spp., a Cyclobacteriaceae spp., a Cystobacteraceae spp. (e.g., a Cystobacter spp.), a Cytophagaceae spp. (e.g., a Cytophaga spp.), a Defluviicoccus spp., a Dehalococcoidales spp. (e.g., a Dehalogenimonas spp., a Dehalococcoides spp.), a Denitratisoma spp., a Derxia spp., a Desulfovibrionales spp. (e.g., a Desulfobacteraceae spp. [e.g., a Desulfocapsa spp., a Desulfatiglans spp., a Desulforegula spp.]), a Desulfoglaeba spp., a Desulfosporosinus spp. (e.g., Desulfosporosinus meridiei), a Desulfotomaculum spp., a Desulfuromonadales spp. (e.g., a Desulfuromonas spp.), a Devosia spp. (e.g., Devosia insulae), a Dickeya spp. (e.g., Dickeya zeae), a Dyadobacter spp., an Ectothiorhodospiraceae spp., an Elusimicrobia spp. (e.g., an Elusimicrobiaceae spp. [e.g., an Elusimicrobium spp.]), an Endomicrobia spp., an Enhygromyxa spp. (e.g., Enhygromyxa salina), an Epilithonimonas spp., an Erwinia spp. (e.g., Erwinia persicina), an Exiguobacterium spp. (e.g., Exiguobacterium undae), a Ferrimicrobium spp., a Fictibacillus spp., a Flavobacteriales spp. (e.g., a Flavobacteriaceae, [e.g., a Flavobacterium spp. such as, for example, Flavobacterium arsenatis, Flavobacterium columnare, Flavobacterium hauense, Flavobacterium johnsoniae, Flavobacterium terrigena]), a Flavisolibacter spp., a Flexibacter spp., a Flindersiella spp., a Fodinicola spp., a Frankia spp., Frigoribacterium spp., a Gaiellales spp. (e.g., a Gaiella spp. [e.g., Gaiella occulta]), a Gallionellaceae spp. (e.g., a Gallionella spp.), a Gemmatimonadales spp. (e.g., a Gemmatimonadaceae spp. [a Gemmatimonas spp.]), a Gemmata spp., a Geoalkalibacter spp., a Geobacillus spp., a Geobacteraceae spp. (e.g., a Geobacter spp.), a Gillisia spp., a Glycomyces spp. (e.g., Glycomyces harbinensis), a Halomonas spp. (e.g., Halomonas muralis), a Haliangium spp., a Herbaspirillum spp. (e.g., Herbaspirillum huttiense), a Holophagales spp. (e.g., a Holophagaceae, spp. [e.g., a Holophaga spp.]), a Humibacillus spp. (e.g., Humibacillus xanthopallidus), a Hydrogenophaga spp. (e.g., Hydrogenophaga palleronii), a Hydrogenophilaceae spp., a Hyphomicrobiaceae spp. (e.g., a Hyphomicrobium spp. [e.g., Hyphomicrobium methylovorum]), a Hyphomonas spp., an Iamiaceae spp. (e.g., an Iamia spp.), an Ideonella spp., an Ignavibacteriales spp. (e.g., an Ignavibacteriaceae spp. such as, for example, an Ignavibacterium spp.), an Ilumatobacter spp., an Intrasporangiaceae spp. (e.g., an Intrasporangium spp. [e.g., Intrasporangium oryzae]), a Jiangella spp., a Kaistia spp., a Kaistobacter spp., a Kallotenuales spp., a Kineococcus spp., a Kineosporia spp. (e.g., Kineosporia mikuniensis), a Knoellia spp., a Kofleriaceae spp. (e.g., a Kofleria spp.), a Kribbella spp. (e.g., Kribbella karoonensis, Kribbella swartbergensis), a Labedella spp., a Labilitrichaceae spp. (e.g., a Labilithrix spp. [e.g., Labilithrix luteola]), a Lactobacillus spp., a Lactococcus spp. (e.g., Lactococcus garvieae), a Lapillicoccus spp. (e.g., Lapillicoccus jejuensis), a Legionellaceae spp., a Leifsonia spp., a Lentzea spp. (e.g., Lentzea albida), a Leptospira spp., a Leptothrix spp., a Leucobacter spp. (e.g., Leucobacter tardus), a Longilinea spp., a Lysinibacillus spp. (e.g., Lysinibacillus sphaericus), a Lysobacter spp., a Marinimicrobium spp., a Marinobacter spp., a Marmoricola spp., a Massilia spp. (e.g., Massilia timonae), a Melioribacteraceae spp. (e.g., a Melioribacter spp.), a Mesorhizobium spp. (e.g., Mesorhizobium loti, Mesorhizobium plurifarium), a Methylibium spp., a Methylobacillus spp. (e.g., Methylobacillus flagellates), a Methylobacteriaceae spp. (e.g., a Methylobacterium spp. [e.g., Methylobacterium adhaesivum]), a Methylocella spp., a Methylococcaceae spp. (e.g., a Methylobacter spp.), a Methylocystaceae spp. (e.g., a Methylocystis spp. [e.g., Methylocystis echinoides]), a Methylosinus spp., a Methyloversatilis spp., a Microbacteriaceae spp. (e.g., a Microbacterium spp. [e.g., Microbacterium kitamiense], a Microcella spp. [e.g., Microcella alkaliphile]), a Micrococcaceae spp., a Microlunatus spp., a Microvirga spp. (e.g., Microvirga aerilata, Microvirga subterranean), a Mycobacteriaceae spp. (e.g., a Mycobacterium spp. [e.g., Mycobacterium sacrum, Mycobacterium salmoniphilum, Mycobacterium septicum]), a Micromonosporaceae spp. (e.g., a Micromonospora spp. [e.g., Micromonospora rhodorangea]), a Modestobacter spp. (e.g., Modestobacter multiseptatus), a Moorella spp., a Myxococcales spp., a Nakamurella spp., a Nannocystaceae spp. (e.g., a Nannocystis spp. [e.g., Nannocystis exedens]), a Neorhizobium spp. (e.g., Neorhizobium huautlense), a Niastella spp., a Nitriliruptor spp., a Nitrosomonadaceae spp. (e.g., a Nitrosomonas spp. [e.g., Nitrosomonas communis, Nitrosomonas ureae]), a Nitrosopumilales spp. (e.g., a Nitrosopumilaceae spp.), a Nitrosospira spp., a Nitrosovibrio spp. (e.g., Nitrosovibrio tenuis), a Nitrospirales spp. (e.g., a Nitrospira spp.), a Nocardiaceae spp. (e.g., a Nocardia spp. [e.g., Nocardia anaemiae]), a Nocardioidaceae spp. (e.g., a Nocardioides spp. [e.g., Nocardioides albus, Nocardioides iriomotensis, Nocardioides islandensis, Nocardioides maritimus, Nocardioides perillae, Nocardia pneumoniae]), a Nocardiopsis spp. (e.g., Nocardiopsis synnemataformans), a Nonomuraea spp. (e.g., Nonomuraea kuesteri), a Nordella spp., a Novosphingobium spp., an Ochrobactrum spp. (e.g., Ochrobactrum haematophilum), an Ohtaekwangia spp., an Olivibacter spp. (e.g., Olivibacter soli), an Opitutaceae spp., an Oryzihumus spp., an Oxalobacteraceae spp., an Oxalophagus spp. (e.g., Oxalophagus oxalicus), a Paenibacillus spp., (e.g., Paenibacillus graminis, Paenibacillus chondroitinus, Paenibacillus validus), a Pantoea spp. (e.g., Pantoea agglomerans), a Paracoccus spp., a Paracraurococcus spp., a Parastreptomyces spp., a Pasteuriaceae spp., (e.g., a Pasteuria spp.), a Pedosphaera spp. (e.g., Pedosphaera parvula), a Pedobacter spp. (e.g., Pedobacter tournemirensis, Pedobacter kribbensis, Pedobacter kwangyangensis), a Pelagibacterium spp. (e.g., Pelagibacterium halotolerans), Pelobacteraceae spp. (e.g., a Pelobacter spp.), a Peptoclostridium spp. (e.g., Peptoclostridium clostridium sordellii), a Peredibacter spp., a Phaselicystidaceae spp., a Phenylobacterium spp., a Phycicoccus spp., a Phycisphaerae spp., a Phyllobacterium spp. (e.g., Phyllobacterium trifolii), a Pigmentiphaga spp., a Planococcus spp., a Planomicrobium spp., (e.g., Planomicrobium novatatis), a Planctomycetes spp. (e.g., a Pirellula spp., such as Pirella staleyi), a Plesiocystis spp., a Polaromonas spp., a Polyangiaceae spp., a Procabacteriacae spp., a Prolixibacter spp., a Promicromonospora spp., (e.g., Promicromonospora sukumoe), a Prosthecobacter spp., a Prosthecomicrobium spp., a Pseudoalteromonas spp., a Pseudoclavibacter spp., (Pseudoclavibacter helvolus), a Pseudolabrys spp., (e.g., Pseudolabrys taiwanensis), a Pseudomonadaceae spp. (e.g., Pseudomonas fluorescens, Pseudomonas flavescens, Pseudomonas protegens, Pseudomonas veronii, Pseudomonas rhodesiae, Pseudomonas koreensis, Pseudomonas moorei, Pseudomonas baetica), a Pseudonocardia spp., (e.g., Pseudonocardia zijingensis, Pseudonocardia carboxydivorans), a Pseudorhodoferax spp., a Pseudoxanthobacter spp., a Pseudoxanthomonas spp., a Ralstonia spp., a Ramlibacter spp., a Reyranella spp. (e.g., Reyranella massiliensis), a Rheinheimera spp., a Rhizobiales spp. (e.g., a Rhizobiaceae spp., a Rhodobiaceae spp.), a Rhizobium spp. (e.g., Rhizobium etli), a Rhizomicrobium spp., a Rhodobacterales spp. (e.g., a Rhodobacter spp.), a Rhodococcus spp. (e.g., Rhodococcus gordoniae, Rhodococcus kroppenstedtii, Rhodococcus wratislaviensis), a Rhodocyclales spp. (e.g., a Rhodocyclaceae spp.), a Rhodomicrobium spp., a Rhodoplanes spp. (e.g., Rhodoplanes elegans), a Rhodopseudomonas spp., a Rhodospirillales spp. (e.g., a Rhodospirillaceae spp.), a Rhodothermus spp., a Rickettsiaceae spp., a Roseateles spp., a Roseomonas spp., a Rubrivivax spp. (e.g., Rubrivivax gelatinosus), a Rubrobacterales spp. (e.g., a Rubrobacter spp.), a Ruminococcaceae spp., a Saccharopolyspora spp. (e.g., Saccharopolyspora gloriosa), a Sandaracinus spp., a Saprospiraceae spp., a Serratia spp. (e.g., Serratia proteamaculans), a Shimazuella spp. (e.g., Shimazuella kribbensis), a Shinella spp. (e.g., Shinella granuli), a Sideroxydans spp. (e.g., Sideroxydans lithotrophicus, Sideroxydans paludicola), a Sinobacteraceae spp. (e.g., a Steroidobacter spp.), a Sinorhizobium spp., a Solibacteraceae spp. (e.g., a Solibacter spp.), a Solirubrobacteraceae spp. (e.g., a Solirubrobacter spp.), a Sorangium spp. (e.g., Sorangium cellulosum), a Sphaerobacterales spp. (e.g., a Sphaerobacteraceae spp. such as, for example, a Sphaerobacter spp.), a Sphingobacteriales spp. (e.g., a Sphingobacteriaceae spp. such as, for example, a Sphingobacterium spp.), a Sphingobium spp. (e.g., Sphingobium herbicidovorans), a Sphingomonadaceae spp. (e.g., a Sphingobium spp. [e.g., S. xenophagum], a Sphingomonas spp. [e.g., S. wittichii]), a Sphingopyxis spp. (e.g., Sphingopyxis macrogoltabida), a Sphingosinicella spp., a Spirochaetales spp. (e.g., a Spirochaeta spp.), a Sporichthyaceae spp. (e.g., a Sporichthya spp.), a Stackebrandtia spp. (e.g., Stackebrandtia nassauensis, a Stella spp., a Stenotrophomonas spp. (e.g., Stenotrophomonas maltophilia), a Stigmatella spp. (e.g., Stigmatella erecta), a Streptacidiphilus spp., a Streptoalloteichus spp., a Streptomycetaceae spp. (e.g., a Streptomyces spp. [e.g., Streptomyces aculeolatus, Streptomyces clavuligerus, Streptomyces fradiae, Streptomyces ghanaensis, Streptomyces glauciniger, Streptomyces hebeiensis, Streptomyces heteromorphus, Streptomyces mashuensis, Streptomyces microflavus, Streptomyces netropsis, Streptomyces phaeochromogenes, Streptomyces roseogriseolus, Streptomyces variabilis, Streptomyces vayuensis, Streptomyces viridodiastaticus, Streptomyces viridochromogenes, Streptomyces xylophagus, Streptomyces xinghaiensis]), a Sulfuricella spp., a Syntrophobacterales spp. (e.g., a Syntrophorhabdaceae spp. such as, for example, a Syntrophobacter spp. [e.g., S. wolinii], a Syntrophorhabdus spp., a Syntrophaceae spp., a Syntrophus spp.), a Taibaiella spp., a Tepidamorphus spp., a Terrabacter spp., a Terriglobus spp., a Terrimonas spp., a Tetrasphaera spp. (e.g., Tetrasphaera elongate), a Thermoanaerobacterales spp. (e.g., a Thermoanaerobacteraceae spp.), a Thermoflavimicrobium spp., a Thermoleophilaceae spp., a Thermomonosporaceae spp., a Thioalkalivibrio spp., a Thiobacillus spp., (e.g., Thiobacillus denitrificans), a Thiobacter spp., a Thiomonas spp., a Thiorhodovibrio spp., a Tolumonas spp., (e.g., Tolumonas auensis), a Variovorax spp., (e.g., Variovorax paradoxus), a Verrucomicrobiales spp., (e.g., a Verrucomicrobia subdivision 3 spp.), a Vibrionales spp., a Woodsholea spp., (e.g., Woodsholea maritima), a Xanthomonadaceae spp., (e.g., a Xanthomonas spp.), a Zoogloea spp., or a Zooshikella spp.

In at least one embodiment, the following can act as an antagonist to at least one of the microbial species listed above, e.g., such as Pseudomonas fluorescens, Pseudomonas mandelii: Streptomyces hygroscopicus, Mycobacterium vaccae, Agrobacterium tumefaciens, Bacillus megaterium, Bacillus amyloliquifaciens, Bacillus subtilus, Bacillus pumilus, a Shingomonas spp., Sphingomonas melonis, an Arthrobacter spp., Agrobacterium rhizogenes, Serratia proteamaculans, Microbacterium testaceum, a Pseudomonas spp., an Erwinia spp., Pantoea agglomerans, Pseudomonas mandelii, a Microbacterium spp., Clostridium saccharobutylicum, Pseudomonas moraviensis, Pantoea vagans, Serratia liquefaciens, Pedobacter kribbensis, Tolumonas auensis, Janthinobacterium lividum, Bacillus racemilacticus, Sporolactobacillus laevolacticus, Brevundimonas mediterranea, Pantoea cloacae, Clostridium acidisoll, Erwinia aphidicola, Bacillus arbutinivorans, Paenibacillus graminis, Pseudomonas veronii, Pseudornonas rhodesiae, Pseudomonas koreensis, Tolumonas auensis, Pseudomonas moorei, Pseudomonas baetica, and/or Pseudomonas protegens.

In certain embodiments, a microbial species that provides insecticidal activity can be added to the microbial inoculant. Suitable microbes can include bacteria or fungi that produce phytochemicals that have insecticidal or insect repelling properties. In some of these embodiments, the microbial species can be a bacterium such as, for example, B. thuringiensis, B. pipilliae Photohabdus luminescens, Pseudomonas entomohpilia, Envinia aphidicola, etc., or a fungus such as, for example, Beaveria bassiana, Lagenidium giganteum, etc. The microbial inoculant composition also can include one or more non-microbial additives. For example, the microbial inoculant composition can include one or more macronutrients or one or more micronutrients such as, for example, carbon, nitrogen, potassium, phosphorus, zinc, magnesium, selenium, chromium, tin, manganese, cobalt, zinc, and/or copper. Suitable macronutrients or micronutrients may enhance the longevity of the bacteria and microbes leading to a longer shelf life. Also, adding a slow growth supporting carbon source (e.g., glycerol, a vegetable oil, lignin, etc.), may be beneficial. This can also function as a stratification media for more anaerobic and aerobic microbes in a single package.

As another example, the microbial inoculant composition can include one or more plant hormone, such as, for example, an auxin. Exemplary suitable plant hormones include auxins such as indole-3-acetic acid (IAA), 4-chloroindole-3-acetic acid (4-CI-IAA), 2-phenylacetic acid (PAA), indole-3-butyric acid (IBA), indole-3-propionic acid (IPA), naphthaleneacetic acid (NAA). Adding a plant hormone to the inoculant composition can provide an initial boost of plant growth and/or establish a faster growth pattern in a field that has, for example, sustained crop damage and is replanted so that the replanted crops need to mature faster than usual.

In at least one embodiment, the plant hormone can be produced by one or more microbes in the microbial inoculant composition. In such embodiments, the microbial inoculant composition can include, for example, Bacillus licheniformis and/or Envinia aphidicola. In this example, the microbial inoculant composition can emit Indole-3-acetic acid (IAA) and/or Indole-3-butyric acid (IBA), which are growth-promoting hormones. The emission of IAA and/or IBA causes the Erwinia aphidicola of the microbial inoculant composition to emit insecticidal enzymes and proteins that reduce the population of aphids and/or other pests of the plant that the microbial inoculant composition is deposited.

As another example, the microbial inoculant composition can include a fertilizing agent. A fertilizing agent may include an organic fertilizing agent or an inorganic fertilizing agent. Exemplary inorganic fertilizing agents may include, for example, nitrogen, phosphorus, potassium, zinc, and/or magnesium. Exemplary organic fertilizers may include, for example, compost, manure, agricultural waste, bone meal, humic extract of peat, and the like or other as known by persons skilled in the art.

As yet another example, the microbial inoculant composition can include one or more adhesive agents to promote the composition adhering to a plant once it is applied to a plant or crop field. One exemplary adhesive agent can include any biocompatible adhesive agent that can be mixed with the microbial inoculant composition and dried onto a seed. As used herein, “biocompatible” refers to an agent that is compatible with the other components of the composition, and not deleterious to the seed or plant to which a formulation that includes the biocompatible component is applied. Suitable adhesive agents include talc, graphite, gum agar, cane sugar, dextrin, commercial potato shellac, starch, or other as known by persons skilled in the art.

In another aspect, this disclosure describes a plant to which any embodiment of the microbial inoculant composition described above is applied. Suitable plants include terrestrial plants, such as, for example, crop plants, trees (deciduous or coniferous), feed plants (e.g., alfalfa), biomass crops, or horticultural plants.

Exemplary crop plants can include wheat, oats, barley, cotton, sugar beets, flax, peanuts, beans, soybeans, potatoes, tomatoes, peppers, corn (especially following sugar beet syndrome), cucumbers, lettuce, cabbage, cauliflower, broccoli, radishes, carrots, celery, jalapeno peppers, okra, Brussels sprouts, watermelon, musk melon, apples, pears, grapes, peaches, oranges, grapefruit, plums, apricots, lemons, avocados, bananas, cassava, sweet potato, pineapple, dates, figs, almonds, walnuts, hazel nuts, pecans, cashews, tobacco, cannabis, oregano, cilantro, sage, saffron, cinnamon, agave, other herbs, or other as known by persons skilled in the art.

Exemplary biomass crop plants can include, poplar trees, switch grass, duck weed, elephant grass, moringa, or other as known by persons skilled in the art.

Exemplary trees to which any embodiment of the microbial inoculant composition can be applied include, for example, cottonwood, willow, birch, poplar, or other as known by persons skilled in the art.

Exemplary horticultural plants can include roses, vines, tubered perennials, petunias, hollyhocks, daffodils, reed sedge, tulips, chrysanthemums, or other as known by persons skilled in the art.

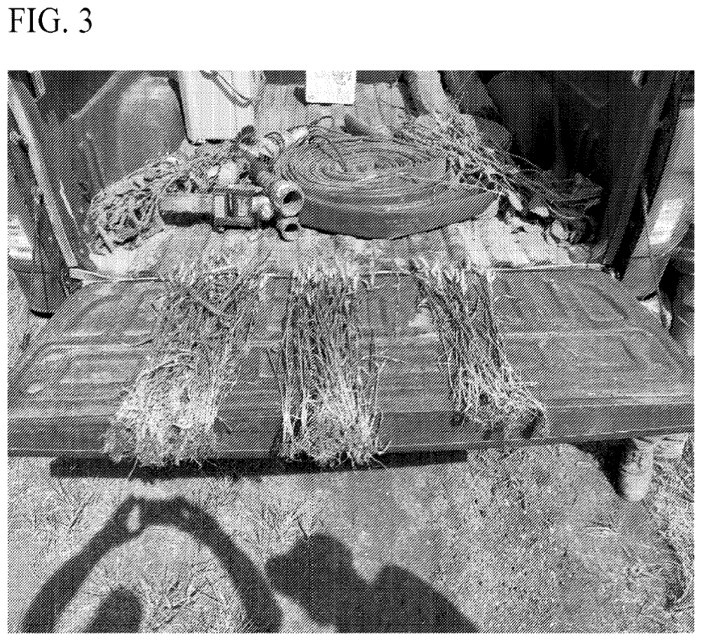

For example, when applied to wheat, the microbial inoculant composition can result in increased stem count, increased tillering, increased head weights, increased seed count, increased size of leaves, increased kernel count, increased kernel weight, increased protein content in the kernel, increased height of the stem, and/or increased overall surface area of the flag leaf. In one example, untreated wheat yielded approximately 50 bushels per acre. A comparable field was treated with amicrobial inoculant composition at the foliar stage, yield was increased to 75 bushels per acre. A comparable field treated at the seed coat stage yielded more than 100 bushels per acre. The wheat treated at the seed coat stage had a 30% increase in the number of kernels, a 20% increase in kernel weight, and a 2% increase in the ratio of protein in the kernel (FIG. 3).

The effect of the microbial inoculant composition can be mitigated to some extent if used in combination with certain fungicides such as, for example, propiconazole. If the fungicide is applied at the manufacturer recommended rate, the efficacy of the microbial inoculant composition can be reduced. For example, when applied to wheat before jointing, the fungicide kills bacteria in the microbial inoculant composition and the effects of the microbial inoculant composition can be negated. If the fungicide is applied to wheat after jointing, one can still see an increase in head count, but increases in leaf size, kernel size, protein ratio, etc. are mitigated.

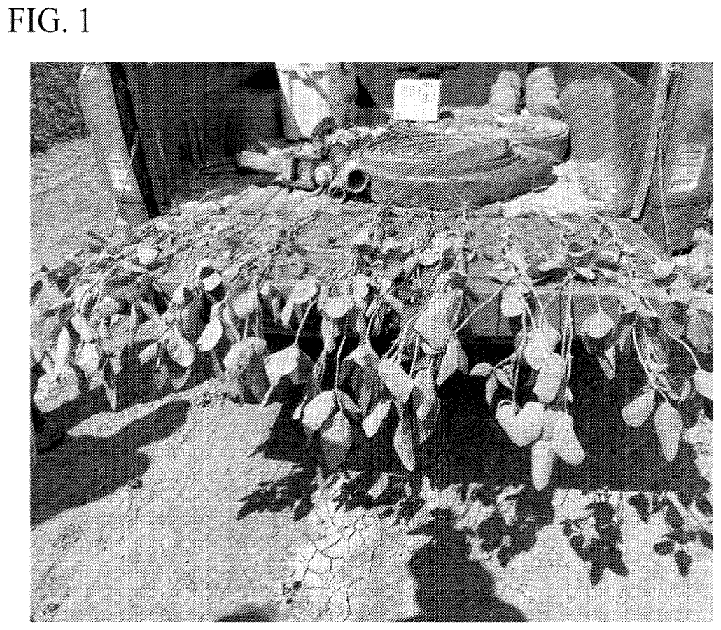

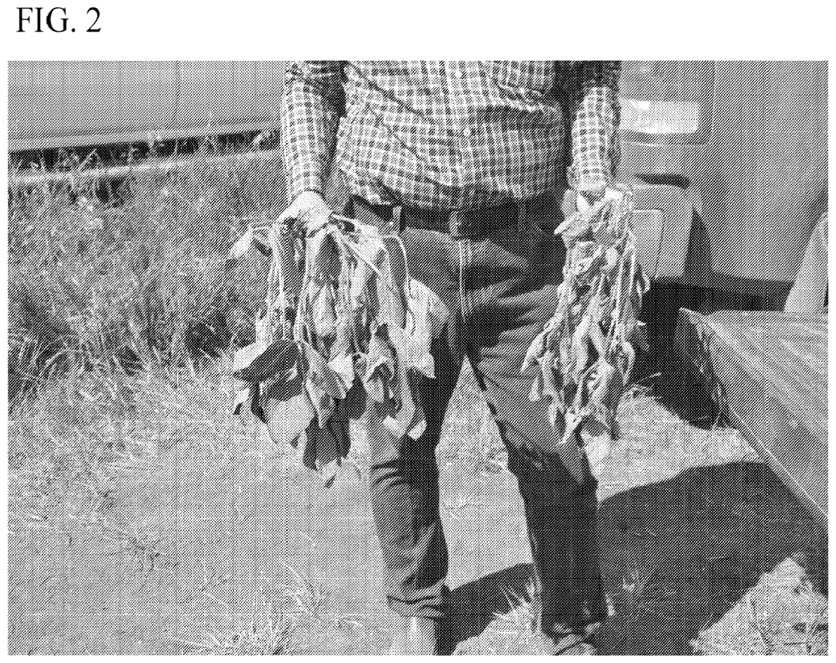

When applied to soybeans, the microbial inoculant composition can result in, for example, increased branching, increased pod count, increased leaf count, increased leaf size, increased number of root nodules, and/or increased size of root nodules (FIG. 1, FIG. 2). In at least one embodiment, the microbial inoculant composition can be applied at an end of a vegetative state of the soybeans. Results of applying the microbial inoculant composition to soybeans can include an increase of anywhere from 4 to 8 bushels per acre. In at least one example result, one field had an increase of 16 bushels per acre. In at least one example method, the microbial inoculant composition is applied to the seed coat, an herbicide is added to damage the leaves of the plant, a Hydra effect occurs, additional herbicide is added to the leaves, and the stalks are broken to further induce the Hydra effect.

When applied to potatoes, the microbial inoculant composition can result in, for example, increased early stage rooting, increased rhizome production, increase the weight of salable potatoes by promoting the first and second set over the third and fourth set, produce darker coloration, increase the above-ground mass of the plant, and/or increase the total weight of tubers produced per acre. In at least one example, the microbial inoculant composition can be applied to potatoes and/or rooted plants, such as sugar beets, onions, carrots, etc. In at least one example of application to onions, a single onion can grow to approximately 3.25 lbs. In contrast, an onion that has not received the microbial inoculant composition can grow to about 0.25 to 0.5 lbs. In addition, in at least one example, onions with the application can have increased volume with less time to get to the onion's normal size, mentioned above. In at least one example, application of the microbial inoculant composition on sugar beets, without splitting, can result in a weight increase of 300%. In at least one example, application of the microbial inoculant composition on sweet potatoes can result in a two-fold increase in size of the sweet potato.

When applied to trees, the microbial inoculant composition can result in, for example, increased height, increased number of leaves in the first year, and/or increased total mass of the tree.

When applied to tomatoes, the microbial inoculant composition can result in, for example, increased flowering, increased bud count, better regeneration after browsing, and/or increased number of tomatoes produced per plant.

When applied to alfalfa, the microbial inoculant composition can result in, for example, increased volume of plant material per acre and/or reduced effects of stress flowering. Reducing the effects of stress flowering allows one to wait longer to cut the alfalfa before it turns woody. In spring, this can allow a farmer to allow the alfalfa to grow longer before it turns woody, thereby allowing the farmer to spend time planting other crops that would otherwise be necessary to cut the alfalfa before it turns woody. Waiting longer between cuttings before the alfalfa turns woody allows one to obtain more tonnage without sacrificing the quality and/or nutritional value of the alfalfa. Also, applying the microbial inoculant composition to alfalfa can result a decrease in the lignin content of the plant as a percentage of total plant biomass. The decreased lignin content can increase the food value of the plant. Applying the microbial inoculant composition also can increase leaf size and/or increase root mass of the plant. Increasing leaf size, like decreasing the lignin content, can increase the food value of the plant. To support the increased photosynthetic surface area that results from the increased leaf size, pants treated with the microbial inoculant composition can exhibit increased root mass, thereby increasing the carbon in the soil. When applied to alfalfa, it may be desirable to reapply the microbial inoculant composition after each cutting.

In at least one embodiment, in response to applying the microbial inoculant composition, alfalfa production can increase by 15 percent in alfalfa production by tonnage. In at least one embodiment, a Rhizobium species and/or minerals including cobalt can be added along with or be added within the microbial inoculant composition. In at least one example, inoculation of alfalfa occurred two weeks prior to cutting, resulting in a 35% increase in tonnage.

The effects of the microbial inoculant composition on alfalfa can be reduced somewhat when there is a zinc deficiency and/or molybdenum deficiency in the soil and/or alfalfa, such as may occur when alfalfa is repeatedly grown in the same field. The mineral deficiency can become a growth-limiting factor. The mineral deficiency can affect the activity of indole-3-acetic acid (IAA) and other growth hormones, affecting the ability of the plant to convert nitrate to ammonium.

When applied to sunflowers, the microbial inoculant composition can result in, for example, increased surface area of flower heads, increased sugars in the flowers, and/or a Hydra effect. In at least one embodiment, a greater than or equal to increase in surface area of flower heads was observed. Increased sugars in the flowers can increase attraction of pollinators and, therefore, increase pollination. The microbial inoculant composition can be added to the sunflower plants in response to the flower heads being at least 3 inches tall, just post-emergence. In at least one example, a Hydra effect including cutting off a first head and growing two replacement heads that are full heads 10½ inches tall was observed. In this example, this can double the yield of sunflower heads.

When applied to bell peppers, the microbial inoculant composition can result in, for example, increased weight of the fruit, increased stem rigidity, and/or increased stem strength.

When applied to corn, the microbial inoculant composition can result in, for example, increased number of kernels per ring and/or increased phosphorus solubility for the plant, thereby mitigating effects of sugar beet syndrome in which an untreated corn plant can manifest stunted plant growth, decreased yield, and/or the corn having a purple appearance. In at least one embodiment, in response to the microbial inoculant composition being applied to corn, a yield increase of one ton to 2.5 tons per acre of dry land silage can result. The application of the microbial inoculant composition is not time dependent; the microbial inoculant composition can be applied at any time from V1 to tassel. When applied to grain corn, in at least one embodiment, within a week of the tassels a 4.8 to 6.8 bushel per acre yield increase can result. In at least one example where corn following sugar beet (CFS) syndrome has occurred, application of the microbial inoculant composition at seed coat or at post-emergence can stabilize phosphorus, leading to the corn overcoming the CFS syndrome effects. CFS syndrome can refer to when corn planting directly follows the planting of sugar beets, which can lead to stunting, shortened internodes, purpling, and/or reduction in vigor.

When applied to small grains, such as wheat barley, oats, rye, etc., applying the microbial inoculant composition prior to a flag leaf can increase the size of the flag leaf, which can, in turn, increase the supply of carbohydrates available to feed the grains. That is, the mass of the small grain can be increased, which can increase tonnage of the small grains. In at least one example, carly application prior to a tiller (e.g., stem) and flag leaf can increase a quantity of stems and increase the weight of the small grain, increasing the tonnage by from 50% to as much as 100%. Also, when applied to the seed coat of small grains, the microbial inoculant composition can increase head count. In at least one example, the microbial inoculant composition can be applied rye or winter wheat in the fall season and again in the spring season.

When applied to cabbage, the microbial inoculant composition can include at least one or more of B. thuringiensis and B. amyloliquifaciens. In at least one example, in response to harvesting cabbage plants that received application of the microbial inoculant composition, the cabbage plants produced multiple heads per plant. In contrast, cabbage plants that did not receive application of the microbial inoculant composition died post-harvest.

When applied to grass, such as prairie grass, lawn grass, sod, etc., the microbial inoculant composition can be applied to both the seed and the grass, increasing leaf size and promoting a darker color, increased growth, and increased root growth that can capture more carbon and/or store increased amounts of carbon in the soil.

When applied to hemp, the microbial inoculant composition can result in, for example, increased height, increased width, increase root size, increased stem girth, increased number of buds, increased size of buds, increased number of seed structures, and/or increased size of seed structures.

When applied to duckweed, the microbial inoculant composition can result in increased root growth. In at least one example, where duckweed can grow up to approximately one (1) inch, application of the microbial inoculant composition can result in growth up to 12 inches. Further, the increased growth of the duckweed can result in increased phosphotransacetylase (pta) biomass as feed. In at least one example, in response to stressing the duckweed plant (such as with dehydration, heat, pH change, etc.) as it is harvested, a breakdown of leucine can occur. The breakdown of leucine can change the amino acid composition and provide a product with lower or no levels of leucine.

When applied to horticultural plants, the microbial inoculant composition can result in, for example, increased growth (whether measured by height, length, or total mass), increased number of blossoms, deeper coloration, faster growing vine, increased size of vine leaves, increased numbers of runners, increased length of runners, and/or tuber perennials carrying over bacteria from the inoculant to subsequent years. In at least one embodiment, application of the microbial inoculant composition to horticultural plants can maintain turgor pressure longer than plants that where the microbial inoculant composition was not applied, causing the plant to maintain aesthetic appeal longer, which can result in greater retail sales and fewer discarded plants.

In at least one embodiment, post-stress damage can occur to any of the above-mentioned plants, trees, and/or crops. This post-stress damage can include hail damage, wind damaged, flooding, etc. As long as the plant, tree, and/or crop is alive, the more the damage, the greater the response due to the microbial inoculant composition. Results of the response can be seen in as little as two weeks. If the microbial inoculant composition is applied prior to the damage, the regeneration of the plant, tree, and/or crop can occur immediately or in close proximity in time to the damage.

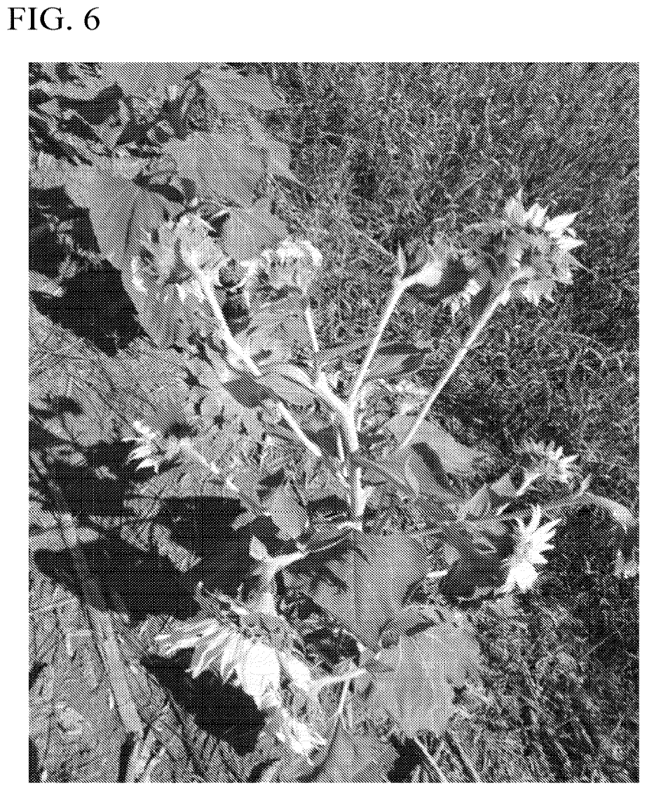

FIG. 6 shows the Hydra effect that results from treating sunflower with a microbial inoculant composition after the sunflower had been mowed. Not only does the sunflower survive the mowing damage, but treatment with the microbial inoculant composition results in increased branching, increased head count, and increased flower surface area compared to a sunflower that is not subjected to mower damage and treatment with the microbial inoculant composition.

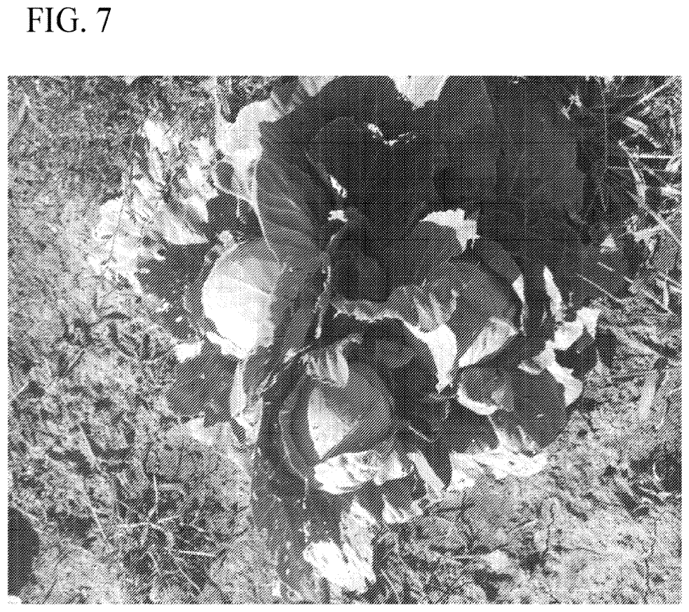

FIG. 7 shows the Hydra effect in cabbage that results from treating cabbage with a microbial inoculant composition after the cabbage had been mowed. Not only does the cabbage survive the mowing damage, but treatment with the microbial inoculant composition results in increased head count and increased head size compared to a cabbage that is not subjected to mower damage and treatment with the microbial inoculant composition.

The microbial inoculant composition can be co-fermented. In at least one example, the microbial inoculant composition includes a mixture of at least one aerobic species and at least one anaerobic species. During co-fermentation, the aerobic microbes typically grow more quickly than anaerobic microbes at first. Eventually, fermentation by the aerobes depletes the fermentation broth of oxygen and produces CO.sub.2. Depletion of oxygen in the broth promotes growth of the anaerobic microbes, while accumulation of CO.sub.2 in the broth slows growth of the aerobic microbes. In this way, a microbial inoculant composition that includes an aerobic species and an anaerobic species can be prepared in a single co-fermentation. In at least one example, the microbial inoculant composition can be aerated to facilitate growth of the Pseudomonas spp. The microbial inoculant composition may be prepared by incubating the microbes in a suitable culture medium at any suitable temperature. A suitable culture medium can include a carbon source (e.g., cane sugar or sucrose), sufficient white vinegar to adjust the pH of the culture medium to no higher than 7.0 (e.g., no higher than 6.8), iron, and a source of potassium (e.g., potassium nitrate).

The microbes may be incubated at a minimum temperature of at least 5° C., such as, for example, at least 10° C., at least 15° C. at least 20° C., at least 25° C., at least 30° C., or at least 40° C. The microbes may be incubated at a maximum temperature of no more than 50° C., such as, for example, no more than 45° C., no more than 45° C., no more than 40° C., no more than 35° C., or no more than 30° C. The microbes may be incubated at a temperature characterized by any range that includes, as endpoints, any combination of a minimum temperature identified above and any maximum temperature identified above that is greater than the minimum temperature. For example, in some embodiments, the microbes may be incubated at a temperature of from 10° C. to 40° C.

The microbial inoculant composition may be prepared by incubating the microbes in a suitable culture medium for a sufficient time to allow growth of both aerobic and anaerobic microbes in the fermentation culture. When a mixture of aerobic microbes and anaerobic microbes are co-fermented, the microbes may be incubated for a minimum of at least 48 hours, such as, for example, at least 72 hours, at least 96 hours, at least 120 hours, at least 144 hours, or at least 168 hours. The microbes may be incubated for a maximum of no more than 240 hours, no more than 216 hours, no more than 192 hours, no more than 168 hours, no more than 144 hours, no more than 120 hours, or no more than 96 hours. The microbes may be incubated for a period characterized by a range having, as endpoints, any combination of a minimum incubation time listed above and any maximum incubation time listed above that is greater than the minimum incubation time.

The microbial inoculant may be applied to seeds, plants, or a field of plants by any suitable method. As described above, the microbial inoculant composition may be formulated with a biocompatible adhesive agent that allows the microbial inoculant composition to be applied to, and adhere to, a seed. Such a formulation can be a folair liquid, seed coating, seed coating hydrogel, etc. The formulation can be mixed into a seeder at planting or can be mixed prior to planting. Alternatively, the microbial inoculant composition may be formulated into with a biocompatible agent that can be applied to seeds and dried. Suitable agents include, for example, dried tapioca, powdered milk, or gum arabic.

Other application methods can involve applying the microbial inoculant composition to one or more tissues of plant, such as, for example, the root, the stem, one or more leaves, or a seed-producing pod. In such cases, the microbial inoculant composition may be applied by any suitable method including, for example, spraying or ampule delivery. The formulation may be sprayed using, for example, a portable spraying unit, hand-held spraying device, irrigation equipment, or aerial spraying. Ampule delivery may be performed manually or using an automated system.

Still other application methods can involve applying the microbial inoculant composition to the soil or seed bed into which seeds will be planted. In these embodiments, the microbial inoculant composition may be applied by spraying or ampule delivery as described immediately above. Alternatively, the microbial inoculant composition may be applied by drip. In some of these embodiments, the microbial inoculant composition can be applied, whether by spray or by drip, while the soil is being seeded.

Still other application methods can include application as a foliar spray, through an irrigation pivot, and as a seed coat. In at least one example, a seed coat media that can hold water can be used to allow the bacteria to live without drying out. In this example, the bacteria can include primarily non-sporulating bacteria that may die when desiccated.

In some cases, a formulation of the microbial inoculant composition can include a predetermined moisture content. The minimum moisture content can be at least 5% such as, for example, at least 6%, at least 7%, at least 8%, at least 9%, at least 10%, at least 15%, at least 20%, at least 25%, at least 30%, or at least 50%.

In some embodiments, a formulation of the microbial inoculant composition can include a sugar (e.g., cane sugar or sucrose) and vinegar (e.g., white vinegar). The sugar can provide a metabolic carbon source. The vinegar can provide an acidic pH and/or an alternative carbon source. As an alternative to, or in addition to, the use of vinegar to regulate pH, the microbial inoculant composition can include Lactobacillus plantarum, as described above, to help maintain an acidic pH once the microbial inoculant composition is applied to the plant.

In other embodiments, a formulation of the microbial inoculant composition can include lactic acid media to provide an acidic pH.

In other embodiments, a formulation of the microbial inoculant composition can include glycerol as a dispersion medium.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Method of Production

Aquatic plants were derived from Long Lake, Codington County South Dakota that were collected (Eurasian Milfoil root and Bullrush Tuber). These aquatic plant tissues were surface sterilized using a 10% bleach solution for 30 seconds. The tissues were then again surface sterilized with a 30% alcohol bath for five seconds then washed with distilled water for 30 seconds to provide material free of epiphytic bacteria and other microbes. This plant material was then macerated and placed in a room temperature seven-gallon incubator with a solution of sucrose, sufficient vinegar to adjust the pH to no more than 6.8, micronutrients (MICROPLEX, Miller Chemical & Fertilizer, LLC, Hanover, Pa.) to get a final iron concentration of 1 ppm, and ¼ teaspoon of potassium nitrate as a nitrogen source. The mixture was allowed to incubate for seven days to allow for sufficient bacteria to grow.

This mixture was then transferred to a plastic tank at room temperature containing between 2500 gallons and 3000 gallons of dechlorinated water in it with 100 pounds of pure cane sugar and two gallons of vinegar, two pounds of nutrient mixture (MICROPLEX, Miller Chemical & Fertilizer, LLC, Hanover, Pa.), and ¼ cup of potassium nitrate. The solution was allowed to incubate to a concentration of bacteria equal to McFarland standard as determined by visual comparison with known standards to a final bacterial concentration of about 3×10.sup.8 CFU/ml. This took approximately 10 days to reach this concentration. This mixture was then decanted into shipping containers for application to the field.

The mixture is applied at a rate of one pint per acre using conventional spraying equipment with an application pressure of 50 psi or less and sufficient droplet size to allow for even plant coverage. The farmers were instructed to use de-chlorinated water (using, e.g., commercially-available dechlorinators such as sodium thiosulfate or tetra sodium salts to remove chlorine or chloramines from the water), or well water and not to mix it with other tank introduced chemicals or herbicides. The farmers also were instructed not to apply additional hormones to the plant once the microbial inoculant was applied.

### Example 2

The microbial inoculant was prepared as described in Example 1. Naphthaleneacetic acid was added to a final concentration of 2 ppm.

### Example 3

The microbial inoculant was prepared as described in Example 1. Bacillus thuringiensis was added to the inoculant to a final concentration of 1.5×10.sup.8 CFU/ml.

### Example 4

The microbial inoculant composition was applied to the plants and early removal of the primary fruit of the plants was performed. A secondary fruit, oftentimes in the form of multiple heads per plant where there was only one, or at least more than the primary fruit, was treated as the primary yield.

### Example 5

The microbial inoculant composition can be put in the presence of gamma-aminobutyric acid (GABA) at 500 ppm in water solution, which causes rapid replication in contrast to plants where the microbial inoculant composition was either not applied or applied but not put in the presence of GABA.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A microbial inoculant composition comprising:
a Pseudomonas mandelii;
an aquatic Delftia spp.; and
a Lactobacillus plantarum.

2. The microbial inoculant composition of claim 1, further comprising an aquatic Pseudomonas spp.

3. The microbial inoculant composition of claim 2, wherein the aquatic Pseudomonas spp. comprises P. moraviensis or P. fluorescens.

4. The microbial inoculant composition of claim 1, further comprising a Clostridium spp.

5. The microbial inoculant composition of claim 4, wherein the Clostridium spp. comprises Clostridium saccharobutylicum.

6. The microbial inoculant composition of claim 1, further comprising a Bacillus spp.

7. The microbial inoculant composition of claim 6, wherein the Bacillus spp. comprises B. megaterium, B. subtilis, or B. licheniformis.

8. The microbial inoculant composition of claim 1, further comprising an aquatic Chryseobacterium spp.

9. The microbial inoculant composition of claim 1, further comprising an aquatic Pseudomonas fluorescens.

10. The microbial inoculant composition of claim 1, further comprising Brevundimonas kwangchunensis, Fictibacillus barbaricus/Bacillus barbaricus, a Prosthecobacter spp., Sphingobacterium multivorum, or a Sphingomonas spp.

11. The microbial inoculant composition of claim 1, further comprising Bacillus megaterium, Bacillus amyloliquifaciens, Bacillus subtilus, Bacillus pumilus, Sphingosinicella microcystinivorans, Pseudomonas chlororaphis, Pseudomonas mandelii, Pseudomonas umsongensis, Arthrobacter ramosus, Streptomyces yogyakartansis, an Arthrobacter spp., Xanthomonas spp., or Chryseobacterium indologenes.

12. The microbial inoculant composition of claim 1, further comprising a yeast strain.

13. A plant comprising the microbial inoculant composition of claim 1 adhered to at least a portion of the plant.

14. A seed comprising the microbial inoculant composition of claim 1 adhered to at least a portion of the seed.

15. A method comprising applying the microbial inoculant composition of claim 1 to a tissue of a plant.

16. A method comprising applying the microbial inoculant composition of claim 1 to a surface of a seed.

17. A method comprising applying the microbial inoculant composition of claim 1 to a seed bed.

18. A method comprising applying the microbial inoculant composition of claim 1 to a field comprising a plurality of plants.

19. A method of producing a microbial inoculant composition, the method comprising:
providing a Pseudomonas mandelii; an aquatic Delftia spp.; and a Lactobacillus plantarum;
providing culture medium comprising:
a carbon source;
sufficient vinegar to adjust the pH to no higher than 6.8;
iron at a concentration of 1 ppm; and
potassium nitrate;
incubating the Pseudomonas mandelii; the aquatic Delftia spp.; and the Lactobacillus plantarum together in the culture medium under conditions effective to allow aerobic fermentation and anaerobic fermentation.

20. The method of claim 19, wherein the Pseudomonas mandelii; the aquatic Delftia spp.; and the Lactobacillus plantarum are incubated at a temperature of at least 15° C.
